(19) United States
(12) Patent Application Publication
(10) Pub. No.: US 2003/0138922 A1
(43) Pub. Date: Jul. 24, 2003

(54) Therapy of stealth virus associated cancers and other conditions using magnetic energy

(76) Inventor: William John Martin, South Pasadena, CA (US)

Correspondence Address: W. JOHN MARTIN, 1634 SPRUCE STREET, SOUTH PASADENA, CA, 91030, US

(21) Appl. No.: 10/058,480

(22) Filed: Jan. 24, 2002

(51) Int. Cl. C12Q 1/70; C12P 1/00; C12N 13/00; C12N 7/00; C12N 7/01

(52) U.S. Cl. 435/173.1; 435/5; 435/41; 435/173.8; 435/235.1

(57) ABSTRACT

A method of causing irreversible damage to stealth virus infected cells, based on exposing the infected cells to magnetic energy. The method of the present application specifically relates to the culturing of stealth viruses from an infected subject and determining the presence of magnetic and/or paramagnetic material in the stealth virus infected culture. Magnetic and/or paramagnetic material can also be found in cultures of bacteria that are infected with the patient's stealth virus. The cultured stealth virus infected cells are tested for susceptibility to cell damage caused by a magnetic field that is not significantly injurious to normal uninfected cells. The presence of magnetic and/or paramagnetic material in the stealth virus culture and the demonstration that exposure to a strong magnetic field can cause damage to stealth virus infected cells, form the basis of therapy for stealth virus infected patients and animals. The therapy comprises exposing stealth virus infected cells within a patient to a magnetic field of sufficient strength to cause damage to cultured virus infected cells. Magnetic energy therapy is especially applicable as therapy for stealth virus associated cancers in which the stealth virus isolated from the cancer patient can be shown to induce the formation of magnetic and/or paramagnetic material.

### CROSS REFERENCE TO RELATED APPLICATIONS

This application is co-pending with the following recently submitted patent applications from William John Martin:

Therapy of Stealth Virus Associated Cancers and Other Conditions Using Light

Therapy of Stealth Virus Associated Illnesses Using Medium Chain Triglycerides

Diagnosing and Monitoring the Therapy of Stealth Virus Infections Based on the Detection of Auto-Fluorescent Material in Hair and in Other Sites.

U.S. Patents

U.S. Pat. No. 5,985,546 Stealth virus detection in the chronic fatigue syndrome William John Martin

U.S. Pat. No. 5,891,468 Stealth virus detection in the chronic fatigue syndrome William John Martin

U.S. Pat. No. 5,753,488 Isolated stealth viruses and related vaccines William John Martin

U.S. Pat. No. 5,703,221 Stealth virus nucleic acids and related methods William John Martin

PCT (Patent Cooperation Treaty)

WO 92/20797 Stealth virus detection in the chronic fatigue syndrome

WO 99/34019 Stealth virus nucleic acids and related methods

WO 99/60101 Stealth Viruses and Related Vaccines

Other Patents

U.S. Pat. No. 5,092,835 Brain and nerve healing power apparatus and method Schurig, et al.

U.S. Pat. No. 5,123,898 Method and apparatus for controlling tissue growth with an applied fluctuating magnetic field. Liboff, et al.

U.S. Pat. No. 5,277,692 Flexible magnetic pad with multi-directional constantly alternating polarity zones Ardizzone V.

U.S. Pat. No. 5,389,981 Eyeglasses having magnets attached thereto for improving the blood circulation of the eyes Riach, Jr

U.S. Pat. No. 5,441,495 Electromagnetic treatment therapy for stroke victim. Liboff, et al.

U.S. Pat. No. 6,004,257 Method for ameliorating the aging process and the effects thereof utilizing electromagnetic energy. Jacobson J I.

U.S. Pat. No. 6,149,577 Apparatus and method for creating a substantially contained, finite magnetic field useful for relieving the symptoms pain and discomfort associated with degenerative diseases and disorders in mammals. Bouldin F, et al.

U.S. Pat. No. 6,293,900 Magnetic face mask Bove, et al

References to Published Articles

Stealth Viruses:

1 Martin W J Chronic fatigue syndrome among physicians. A potential result of occupational exposure to stealth viruses. Explore 2001; 10 (5): 7-10.

2 Martin W J. Stealth Viruses. Explore 2001; 10 (4): 17-19.

3 Durie G M, Collins R. Martin W J. Positive stealth virus cultures in multiple myeloma. A possible explanation for neuropsychiatric co-morbidity. Presented at the Am. Soc. Hematology annual meeting October 2000.

4 Martin W J. Chemokine receptor-related genetic sequences in an African green monkey simian cytomegalovirus-derived stealth virus. Exp Mol Pathol. 2000; 69:10-6.

5 Martin W J. and Anderson D. Stealth virus epidemic in the Mohave Valley: severe vacuolating encephalopathy in a child presenting with a behavioral disorder. Exp Mol Pathol. 1999; 66:19-30.

6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin W J. Bacteria-related sequences in a simian cytomegalovirus-derived stealth virus culture. Exp Mol Pathol. 1999; 66:8-14.

8 Martin W J. Stealth adaptation of an African green monkey simian cytomegalovirus. Exp Mol Pathol. 1999; 66:3-7.

9 Martin W J. Cellular sequences in stealth viruses. Pathobiology 1998; 66:53-8.

10 Martin W J. Detection of RNA sequences in cultures of a stealth virus isolated from the cerebrospinal fluid of a health care worker with chronic fatigue syndrome. Case report. Pathobiology. 1997; 65:57-60.

11 Martin W J. and Anderson D. Stealth virus epidemic in the Mohave Valley. I. Initial report of virus isolation. Pathobiology. 1997; 65:51-6.

12 Martin W J. Simian cytomegalovirus-related stealth virus isolated from the cerebrospinal fluid of a patient with bipolar psychosis and acute encephalopathy. Pathobiology. 1996; 64:64-6.

13 Martin W J. Stealth viral encephalopathy: report of a fatal case complicated by cerebral vasculitis. Pathobiology. 1996; 64:59-63.

14 Martin W J. Genetic instability and fragmentation of a stealth viral genome. Pathobiology. 1996; 64:9-17.

15 Martin W J. Severe stealth virus encephalopathy following chronic-fatigue-syndrome-like illness: clinical and histopathological features. Pathobiology. 1996; 64:1-8.

16 Martin W J. Stealth virus isolated from an autistic child. J Autism Dev Disord. 1995; 25:223-4.

17 Gollard R P, Mayr A., Rice D A, Martin W J. Herpesvirus-related sequences in salivary gland tumors. J Exp Clin Cancer Res., 1996; 15: 1-4.

18 Martin W J. and Glass R T. Acute encephalopathy induced in cats with a stealth virus isolated from a patient with chronic fatigue syndrome. Pathobiology. 1995; 63:115-8.

19 Martin W J, et al. African green monkey origin of the atypical cytopathic ‘stealth virus’ isolated from a patient with chronic fatigue syndrome. Clin Diag Virol 1995: 4: 93-103.

20 Martin W J. Stealth viruses as neuropathogens. CAP Today. 1994; 8: 67-70.

21 Martin W J. et al. Cytomegalovirus-related sequence in an atypical cytopathic virus repeatedly isolated from a patient with chronic fatigue syndrome. Am J Pathol. 1994; 145: 440-51.

Other Cited References

Cutnell J D and Johnson K W. Physics. John Wiley and Sons, Inc. New York, 1992

Iannini R. Mesmer and mesmerism. Med Secoli 1992;4:71-83

Jacobson J I. Et al. Low-amplitude, extremely low frequency magnetic fields for the treatment of osteoarthritic knees: a double-blind clinical study. Altern Ther Health Med 2001;7:54-64, 66-9

Man D et al. The influence of permanent magnetic field therapy on wound healing in suction lipectomy patients: a double-blind study. Plast Reconstr Surg 1999;104:2261-6.

Ramey D W. Analysis: Magnetic and Electromagnetic Therapy. The Scientific Review of Alternative Medicine. Prometheus Books 1998

Vsevolodov N. Biomolecular Electronics: An Introduction via Magnetic and/or Paramagnetic Proteins. Birkhauser, Boston 1998

Weinberger A et al. Treatment of experimental inflammatory synovitis with continuous magnetic field. Isr J Med Sci 1996; 32: 1197-201

### STATEMENT REGARDING FEDERALLY SPONSORED RESEARCH OR DEVELOPMENT

No Federal funding was received in support of the research covered in this patent application.

Reference to Sequence Listing, a Table, or a Computer Program Listing Compact Disk Appendix

None provided.

### BACKGROUND OF THE INVENTION

The present invention relates to therapy of virus infections, in which the virus belongs to a group of atypically structured, non-inflammation-inducing viruses, for which the inventor has coined the term stealth viruses. The patent application relates particularly to the therapy of cancers in which the malignant cells are infected with a stealth virus. Methods for the detection and characterization of stealth viruses are covered in U.S. Pat. Nos. 5,985,546; 5,891,468; 5,753,488; and 5,703,221. Although initially identified in association with neuropsychiatric illnesses, including chronic fatigue syndrome, stealth viruses can also commonly be cultured from cancer patients and can be detected within tumor tissues. (Stealth virus-related references are listed in this application and are numbered 1-21. All cited patents, co-pending patent applications and the entire list of stealth virus publications are incorporated herein by reference).

The basis of the present invention is the discovery that certain stealth viruses lead to marked intracellular accumulation of a diverse range of particulate materials, some of which possess and/or are responsive to magnetic energy. Magnetism is an energy force that originates from certain materials, known as magnets, because of a dysequilibrium in the natural spinning motions of positive and negative charged sub-atomic particles. It is also a fluctuating component of electromagnetic radiation and accompanies electricity, light, and radio-waves, etc. The magnetic energy of a magnet is derived from unbalanced electrons spinning on their own axis. For most atoms there are an equal number of electrons spinning in opposing directions. This leaves no net magnetic energy emission. In materials composed of atoms with an unpaired electron, the atoms are generally present in a random configuration. The magnetic energies coming from each of the atoms in these material are, therefore, also directed in a randomized fashion, with little or no overall measurable magnetic output from the material. For only a few substances, and especially iron, the atoms can be oriented in a highly coordinated manner, such that the magnetic energy originating from the unpaired electron in each atom will tend to move in the same direction. The aggregate directional magnetic energy from these substances forms a magnetic field around the material. The field extends from one end of the material to an opposite end. These ends are called “poles” and are designated north or south, depending on their relationship to the net magnetism of the earth. When magnets are in close proximity, there is an attraction force between the north and south poles of the different magnets. There is a corresponding repelling force between the north poles, and also between the south poles, of the magnets.

A strong magnetic field can align the orientation of the atoms within certain types of different materials. If the atoms in these materials have unpaired electrons, the material can become magnetic; at least for as long as it stays in the magnetic field of a strong magnet. Materials that become magnetized in a strong magnetic field can be either attracted towards the magnet (paramagnetic) or repelled from the magnet (diamagnetic). For this specification, I will use the term paramagnetic to cover both paramagnetic and diamagnetic materials.

Most magnets are also paramagnetic since their strength can be enhanced by being placed within the magnetic field of a stronger magnet. The added magnetic strength dissipates when the inherently weaker magnets are removed from the stronger magnetic field. The strength (or flux) of a magnet can be measured as the attraction force per unit area at the magnet's poles. The unit of this measurement is called gauss. A typical magnet used in a high school science class will be approximately 50 gauss. Small industrial magnets can range from 200-15,000 gauss. The surface of the earth has a magnetic field strength of approximately 0.5 gauss. Magnetic strength diminishes as a cubic function of the distance from the magnetic poles (Cutnell J D and Johnson K W. Physics. John Wiley and Sons, Inc. New York, 1992).

As noted above, magnetic energy can be a component of, and can interact with, other forms of energy. The best understood relationship exists between magnetism and electricity. Thus a fluctuating magnetic field always accompanies the flow of electrons in an electric current. Conversely a fluctuating magnetic field can induce electrons to move along a conducting material. This can create electricity. Magnetism can also interact with radio-waves. An example of this interaction is sun's interference with radio-transmissions during magnetic solar flares. On the medical front, magnetism induced alignment of various atoms within organ structures can be altered by a pulsed radio-wave. The return speed of the atoms to their magnetism induced alignment is measured in magnetic resonance imaging (MRI) based diagnostic imaging procedures.

An enormous folklore has arisen on the possible beneficial effects of magnetism on human health. The healing power of magnets was widely promoted by Franz Anton Mesmer in the 18^{th} century (Iannini R. Mesmer and mesmerism. Med Secoli 1992; 4: 71-83). He proposed that the human body was a magnet, with north and south poles and that diseases could arise from a misalignment of the poles. He and subsequent followers believed they could help align the magnetic forces using magnets, electricity and massage. From these beginnings, the art of chiropractic therapy evolved with its increasing emphasis on physical manipulation, rather than on magnetic or electrical therapy. There have been occasional reports of the use of magnets to promote wound healing, possibly by the magnetic field enhancing blood flow (for example; Man D et al. The influence of permanent magnetic field therapy on wound healing in suction lipectomy patients: a double-blind study. Plast Reconstr Surg 1999;104:2261-6). Magnets have also been used as possible therapy for arthritis (for example; Weinberger A et al. Treatment of experimental inflammatory synovitis with continuous magnetic field. Isr J Med Sci 1996; 32: 1197-201 and Jacobson J I. et al. Low-amplitude, extremely low frequency magnetic fields for the treatment of osteoarthritic knees: a double-blind clinical study. Altern Ther Health Med 2001; 7: 54-64, 66-9).

Many other studies have refuted claims of significant health benefits from the use of either permanent fixed magnets or electricity driven fluctuating magnetic fields. (Ramey D W. Analysis: Magnetic and Electromagnetic Therapy. The Scientific Review of Alternative Medicine. Prometheus Books 1998). Proponents of alternative medicine have nevertheless persisted in making claims that exposure to magnets can improve a wide variety of medical conditions, especially those causing pain. Numerous magnetic products such as bracelets, necklaces, shoe insoles, and even mattresses are available from health stores and are being promoted for a wide variety of minor health ailments. Although there is no scientific foundation for the claims, one will not uncommonly hear of a patient who feels that he or she has benefited from using a magnet to treat a minor chronic medical condition.

The therapeutic role of magnets for major illnesses, particularly cancer, is currently discounted by virtually all of mainstream medicine, and is rarely addressed even by the most avid proponents of alternative medical practice. A major difficulty in even pursuing studies in this area is the lack of any scientific rationale as to how magnets could be selectively working on seriously diseased tissue. The present patent application provides such a rationale. It demonstrates that magnetism can kill stealth virus infected cells and teaches how magnetism can be applied to the therapy of stealth virus associated cancers and other conditions in both human and animals.

### BRIEF DESCRIPTION OF THE DRAWINGS (FIGURES)

FIG. 1 A low power photomicrograph showing three pigmented cell clusters that developed in a strongly positive stealth virus culture. A clearly colored red thread-like structure passed through the largest of the three cell clusters. A single cell can be seen emerging from the upper left hand side of the largest cell cluster. This photograph was taken immediately before the culture tube was placed between two magnets.

FIG. 2. Photomicrograph of the same cell cluster as is shown in FIG. 1. This photo was taken 15 minutes after exposure of the culture to the magnetic field provided by two magnets placed on opposing sides of the culture tube. The single cell that had emerged from the upper left hand side of the large cell cluster has retracted.

FIG. 3. Photomicrograph of the same cell cluster as is shown in FIG. 1. This photo was taken 12 hours after exposure to the magnetic field provided by two magnets placed on opposing sides of the culture tube. The lower cluster has become detached from the glass surface and now overrides the lower end of the large cell cluster. The clusters have also contracted in size by approximately 20%. They were also noticeably more pigmented than at the start of the experiment.

FIG. 4. Photomicrograph of the same cell cluster as is shown in FIG. 1. One of the magnets was removed at 12 hours leaving a magnetic field provided by a single magnet. Within 2 hours of using the single magnet, the top cluster had become detached from the thread and moved into a position overlaying the upper left hand side of the large cell cluster.

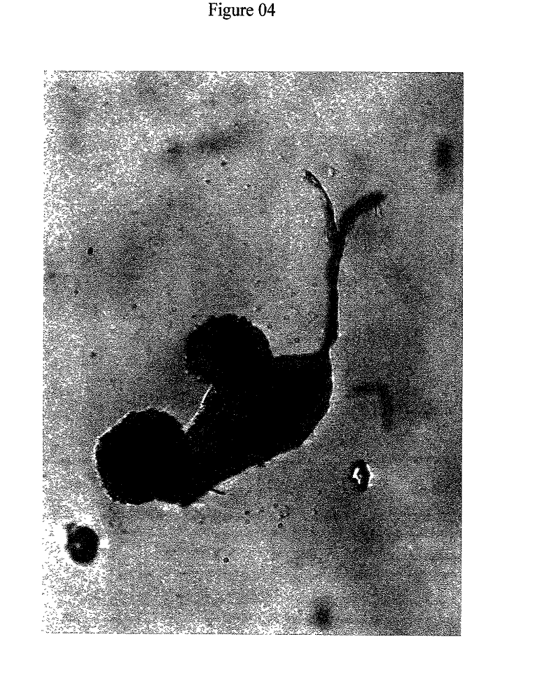

FIG. 5. A dark field black and white photomicrograph of the same cell cluster as is shown in FIG. 1. This photo was taken at the end of the experiment to show the marked auto-fluorescence emanating from the clusters and from the thread-like structure. The clusters, and particularly the thread-like structure, also gave a transient red afterglow after switching off the microscope light.

FIG. 6. A low power photomicrograph showing two adjacent cell clusters that developed in another stealth virus culture. Relatively normal appearing elongated cells can be seen around the edges of both of the cell clusters. The cell clusters were heavily pigmented. A curved thread-like structure can be seen passing through one of the cell clusters.

FIG. 7. A high power photomicrograph of the edge of one of the cell clusters seen in FIG. 6. It shows relatively healthy appearing cells, especially just beneath the U shaped curvature of the thread-like structure.

FIG. 8. A high power photomicrograph of the region between the two cell clusters shown in FIG. 6. This photomicrograph was taken 3 hours after the culture tube was placed in a magnetic field provided by a pair of magnet. Many of the cells that had previously appeared to be relatively healthy have became rounded and were beginning to retract from each other. They appeared to be undergoing significant damage as a result of exposure to the magnetic field.

FIG. 9. A low power photomicrograph taken after an additional 9 hours exposure to a magnetic field that since the 3rd hour was being provided by a single magnet. The thread-like structure had flipped over from its original position. The diameters of the pigmented areas within each of the clusters had also retracted by approximately one third of what they were at the start of the experiment. Most of the cells surrounding the clusters continued to show a markedly unhealthy appearance.

FIG. 10. This high power photomicrograph was taken from the same culture as that used in the experiment depicted in FIGS. 6-9. While most of the culture continued to show extensive cell damage and degeneration, occasional clusters were present in which a few remaining cells had begun to proliferate. It seemed as if certain cells in the culture had been essentially unaffected by the magnetic field. This particular photomicrograph was taken 1 day after the 12 hour prior exposure to the magnetic field. At the time the experiment started, the small cell cluster was presumably surrounded by several rows of proliferating cells. While most of the cells surrounding the cell cluster had apparently died and detached from the glass surface of the culture tube, a few cells had clearly survived and were seemingly resuming proliferation as suggested by this photo.

FIG. 11. Photomicrographs showing the influence of a hand held magnet on the positioning of a floating, heavily pigmented, cell cluster from a stealth virus culture. The cell cluster was originally at the center of the incomplete X marking seen in the field of the microscope. The cell cluster moved towards the upper right side of the X marking in response to a magnet being placed above the tube and slightly to the upper right of center (top photo). The positioning of the magnet was changed such that it next approached the cell cluster from the lower left hand side. This caused the cell cluster to rotate180° and to move across the X marking to the lower left side (middle photograph). This directional movement continued as the magnet was slowly withdrawn (lower photograph).

### BRIEF SUMMARY OF THE INVENTION

The invention teaches a method of treating stealth virus infected patients by causing irreversible damage to stealth virus infected cells using magnetic energy. The method is based on the discovery that stealth virus infected cells can produce abnormal, aggregated, intracellular and extra-cellular materials, and that some of this material can be magnetic and/or paramagnetic. Activation of this material by exposure to a strong magnetic field provides a means of inducing cell damage to the stealth virus infected cells. The method of the present application specifically relates to the culturing of stealth viruses from infected patients and determining the presence of magnetic and/or paramagnetic materials in the stealth virus infected cultures. These materials can also be sought in bacteria infected with the patients' stealth viruses. The infected cells can be further tested for their sensitivity to the cellular destructive effect of the magnetic energy, provided by a strong magnetic field. The detection of magnetic and/or paramagnetic materials in the stealth virus culture from a patient forms the basis of exposing infected cells in the patient to a therapeutic magnetic field. The specific purpose of the therapy is to kill the viral infected cells. This approach is particularly applicable to the therapy of stealth virus associated cancers, in which the stealth virus isolated from the cancer patient can be shown to induce the formation of magnetic and/or paramagnetic materials. This method has wider applications, including the destruction of stealth viruses infected cells found in many other disease states, in both humans and animals. It can also be applied to destroying suspected stealth virus infected cells in blood used for transfusion and in tissues used for transplantation. It may also be utilized as a method to destroy stealth virus infected bacteria that are producing magnetic and/or paramagnetic materials.

### DETAILED DESCRIPTION OF THE INVENTION

The present invention provides a method to selectively damage stealth virus infected cells within a patient. The method is based on determining the presence of magnetic and/or paramagnetic materials in stealth virus infected cells, and in the extra-cellular materials produced in stealth virus cultures. These materials will respond to an externally applied strong magnetic field. The response includes the induction of cellular damage. The ability of a magnetic field to damage stealth virus infected cells in vitro can be readily determined microscopically. The finding of magnetic and/or paramagnetic materials in a stealth virus culture from a patient, can lead to the confident use of a strong magnetic field as a means of causing damage to stealth virus infected cells within the patient. This method has therapeutic applications in the therapy of stealth virus infected human and animal subjects.

As used herein stealth viruses refers to infectious agents that will cause a characteristic vacuolating cytopathic effect (CPE) in human and animal tissue culture cells using procedures described for the cultivation of stealth viruses. These procedures have been provided in various patents and publications relating to stealth viruses. Essentially, it is possible to demonstrate the presence of a stealth virus in peripheral blood or tissues of a stealth virus infected patient, by following tissue culture procedures that will allow for the expression of a stealth virus induced CPE. A suitable procedure is as follows: Mononuclear cells are separated from 8 ml of whole blood, collected in an acid citrate dextrose (ACD) blood vacutainer tube using Ficoll Paque (Pharmacia, NJ) density centrifugation. After washing the mononuclear cells in phosphate buffered saline, they are re-suspended in 2 ml of serum free, X Vivo-15 medium (BioWhittaker Inc., MD). The cells are aliquoted into two vials, each of which is stored frozen until testing. The supernatant and the cell pellet from a lightly centrifuged thawed vial are each added to culture test tubes containing MRC-5 human fibroblasts (BioWhittaker Inc., MD), in 3 ml of serum free X Vivo-15 medium. The culture tubes are placed on a slowly rotating wheel (Cel-Gro, Lab Line, Medford Ill., 4 minutes per rotation) in a 36.5° incubator. The tubes are examined regularly using an inverted phase contrast microscope. The appearance, rate of progression and host range of the CPE caused by stealth-adapted viruses are quite dissimilar from those caused by any of the commonly encountered conventional human cytopathic viruses, including human herpes simplex viruses, cytomegalovirus, Epstein-Barr virus, human herpesvirus-6, varicella-zoster virus, human adenoviruses, measles virus, or enteroviruses.

In the case of MRC-5 human fibroblast indicator cells, the normal spindle shaped, translucent, closely packed cells become enlarged, rounded and tend to fuse into small, and later into larger, three dimensional cell syncytia and clusters. The cellular cytoplasm displays a vacuolated, lipid-laden-like appearance. With time, and especially in larger cell clusters, an additional accumulation of yellow-brown to golden-black, fine and/or coarse pigmentation, can be readily seen within affected cells, and sometimes in the culture supernatant. Even more striking is the formation of long extra-cellular pigmented thread, ribbon and tube-like structures in many of the longer-term cultures. Abnormal appearing particulate materials are also a conspicuous feature of some of the abnormal cells seen in various tissue biopsies of stealth virus infected humans and animals (5,13,15,18).

While there are major overall similarities between stealth virus cultures from different patients, there are also many subtle differences, especially in terms of the extent, coarseness, color and types of particulate intracellular and extra-cellular materials, and in the tendency to form smaller or larger cell syncytia and cell clusters. The rate of progression of the CPE can also differ between cultures, but in most cases can be clearly promoted by frequent (every 2-3 days) replacement of the tissue culture medium. The capacity to culture stealth viruses from patients allows for the detailed characterization of the intracellular and extra-cellular particulate and other extraneous materials present in the stealth virus cultures from different patients.

The underlying molecular hypothesis for the origins of stealth viruses is the initial loss of specific genes that, if present, would code for the major viral components required for effective immune recognition of the viral infected cell by anti-viral cytotoxic T lymphocytes (CTL). For many viruses, relatively few components act as effective targets for cell-mediated immunity (8). For example, in the case of cytomegaloviruses, the majority of anti-viral CTL are directed against a protein termed UL83 and most of the remaining CTL are directed against either the UL55 gene product or the immediate-early antigen. The UL83 and UL55 genes were not detected in a stealth virus derived from an African green monkey simian cytomegalovirus (SCMV), while the immediate-early gene showed numerous mutations (8). Stealth viruses appear to retain and/or regain their replicative and cell damaging activities by incorporating additional genes from other viruses, infected cells (9) and even from bacteria (7). This has been clearly shown for the prototype SCMV-derived stealth virus. Many of the viral, cellular and bacterial genes identified in cultures of this particular stealth virus show evidence of having undergone significant mutations that may well influence their biochemical and biophysical properties.

As used herein, magnetic materials refer to matter that has both a north and a south magnetic pole. This can be shown by attraction of one end of the material towards the north magnetic pole of a known magnet, and the repulsion of this end of the material from the magnet's south pole. The other end of the material will be repulsed by the north end of a known magnet, and attracted to a magnet's south pole. The term paramagnetic refers to a material which does not possess intrinsic magnetic activity when it is not exposed to a strong magnetic field, yet will become magnetized when exposed to a strong magnetic field. A paramagnetic material becomes attracted to whichever is the closest pole of the magnet generating the magnetic field. (Diamagnetic materials differ from paramagnetic materials in that once magnetized they are repulsed from whichever is the closest pole of the magnet generating the magnetic field). As noted above, in this application, the term paramagnetic is being used to imply both paramagnetic and diamagnetic materials.

The term strong magnetic field is used to describe the magnetic energy surrounding a typical industrial strength magnet. The actual magnets chosen to perform the studies reported in this specification were composed of neodymium-iron-boron (Nd2Fe14B) and are commonly referred to as rare earth magnets. They were slightly curved rectangular shaped and measured 1-¾″×1-7/16″×¼.″ The poles were on the flat surfaces with a magnetic strength of 850 gauss, providing a pull from their edge against a steel surface of approximately 15 lbs. They were purchased from C and H Sales Company, Pasadena Calif. 91107, catalog number MAG9850. The same store sells magnets ranging from 200-2000 gauss strength. The magnets chosen are considered strong, but not that strong as to pose a problem in their safe handling near metal objects.

The focus of this patent application has been to explore the effects of a strong magnetic field on the abnormal cellular products accumulating in stealth virus infected cells. I had shown that the abnormal cellular material could be auto-fluorescent. This implied that it had electrons that could exist at altered energy levels. I also knew that certain molecules can absorb different forms of energy and convert the incoming energy into other forms of energy and/or into chemical processes (Vsevolodov N. Biomolecular Electronics: An Introduction via Magnetic and/or Paramagnetic Proteins. Birkhauser, Boston 1998). As implied by the title of this book, while magnetism is commonly associated with metals such as iron, it can be a property of various protein and non-protein structures. I, therefore, decided to test whether some of the abnormal intracellular and extra-cellular materials accumulating in stealth virus cultures would respond to a strong magnetic field. I also knew that toxic free radicals can be produced, and that electrons and ions can be displaced, in response to energy consumption and conversion reactions mediated by complex molecules. I, therefore, wanted to further see if a strong magnetic field could affect the viability of stealth virus infected cells.

I conducted a series of experiments similar in principle to those described in a co-pending patent application, “Therapy of Stealth Virus Associated Cancers and Other Conditions Using Light,” The difference was in the use of a strong magnetic field instead of light. Specifically, I selected several stealth virus positive culture tubes that were beginning to show the outgrowth of relatively normal appearing MRC-5 cells from the edges of cell clusters that had formed as a result of the stealth virus infection. The cell clusters contained pigmented, particulate material, which typically forms in stealth virus cultures. The culture tubes were placed horizontally in a Styrofoam holder with either a single magnet, or two opposing magnets adjacent to the side(s) of the tube and separated from the side by approximately 1 cm of Styrofoam. The tubes were incubated at 36.5° C. for varying periods of time. The culture tubes were examined at regular intervals and changes to a previously identified cell cluster noted. As illustrated below, I observed the following changes: i) A definite loss of viability of cells emerging from the cell clusters. Instead of being elongated with a healthy appearing cytoplasm and distinct nucleus, they became rounded, losing a clearly definable nucleus and acquiring a more marked pigmentation. ii) The pigment in the clusters also became more intense with subtle changes in the overall pattern. iii) The clusters would shrink in size and occasionally lift off from the glass surface of the tube. iv) There was an increase in the amount of free floating granular pigmented material. and v) The number of ribbon-like structures increased and also showed signs of progressive detachment from the cell clusters. Significant changes were seen within 1-2 hours and generally progressed over a 12 hour period of observation. With some cultures a single magnet appeared to induce more rapid toxicity that using two magnets with opposing different poles. Control cultures were similarly exposed to either one or two magnets. Only after 12 hours, were there discernable, but minor changes that mainly affected the edges of the cell monolayer. Some of the normal cells would retract and change into small shrunken cells. Overall, the vast majority of the cells in the normal culture would remain essentially unaffected.

In the next series of experiments, I tested several small freely floating, pigmented clusters for their response to an approaching magnet. Especially in the cultures that had been previously exposed to a magnetic field, I could find the occasional floating cluster that would move in unison with the magnet. The movement of different clusters could be either towards or away from the approaching magnet. By changing the end of the magnet, I could reverse the effect seen between attraction and repulsion. This striking demonstration of actual magnetism of the cluster was only seen with an occasional pigmented cluster, but when seen it was unmistakable. I could also confirm it with some of the floating pigmented clusters present in cultures that had not been previously exposed to a strong magnetic field. The strength of the magnets used in these studies has been rated at only 850 gauss. Although this is a reasonable strength, far stronger magnets of over 10,000 gauss are readily available. Also, I could not approach closer than approximately 1 cm from the floating clusters because of the glass culture tube. It is probable, therefore, that more of the clusters could be shown to be magnetic and/or paramagnetic if I had used a stronger magnet and directly applied it to the clusters.

A feature of certain stealth viruses is the capacity to be propagated in, and to induce morphological and biochemical changes in bacteria and even to cause bacterial death (7). I investigated whether bacteria exposed to stealth virus infected tissue culture medium could be influenced by a magnetic field. I chose a thioglycolate broth culture of the bacteria that I had infected with a stealth virus cultured from a patient (CM) who has a breast cancer. Normally the bacteria will grow only in a 1-2 cm band beginning at approximately 1 cm below the surface of a broth. I placed a magnet 5 mm from the side of the bacteria culture tube, extending from the region of the banded bacteria growth to halfway down the tube. After 12 hours, it was clear that the bacteria had begun to partition slightly, both towards and away from the magnet. Thus the density of bacterial growth was diminished in the section of the tube midway from the side of the tube facing the magnet and the side of the tube most distant from the magnet. More easily defined was the spreading of bacteria down into the tube beneath the usual band of growth. This result was taken as evidence that the bacteria probably contained magnetic and/or paramagnetic material presumably coded by a patients' stealth-adapted viruses.

As described in a co-pending patent application (Therapy of Stealth Virus Associated Cancers and Other Conditions Using Light), the patient (CM) had chosen not to have breast surgery and has lived with a histological confirmed infiltrating breast cancer of her right breast for over 9 years. She had actually tried using a magnet on her breast cancer approximately one year ago. She recalled that it had caused her tumor to ache within 2-3 days of placing the magnet close to her tumor. The aching was a recurring pressure sensation that necessitated her removing the magnet. The aching subsided within a day or two or her removing the magnet. I requested that she might repeat the study using one of the magnets that I had used in the above study. She placed the magnet in a small packet that she sewed into her brassiere. The magnet directly overlaid her breast cancer. Within 3 hours of wearing the brassiere, she noted a significant, although slight, burning sensation within the tumor. It lasted for 2 hours and then gradually subsided, only to return several hours later. The significant, although slight, slight pain and burning sensations persisted on and off throughout the next day. Plans are underway to obtain Institutional Review for the continuation of this type of study.

### EXAMPLES AND ILLUSTRATIONS

The findings described in this application have been derived mainly from detailed examinations performed on stealth virus cultures. The culturing of stealth viruses is not yet a generally accepted method of research and/or clinical testing. Detailed documentation of the typical stealth virus CPE is provided in the co-pending patent application “Therapy of Stealth Virus Associated Cancers and Other Conditions Using Light.” The figures from this application should be reviewed as a background to the following descriptions.

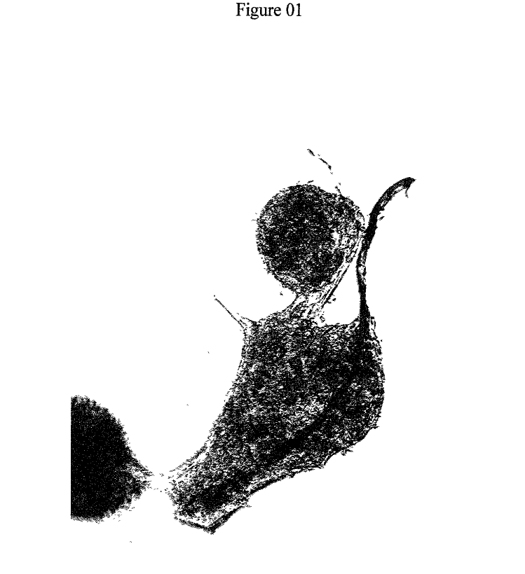

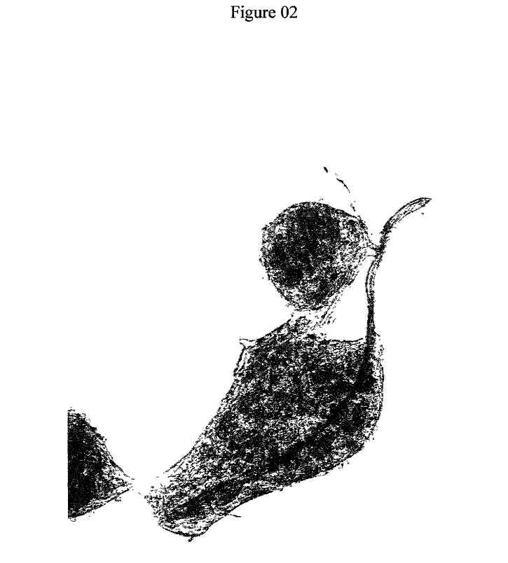

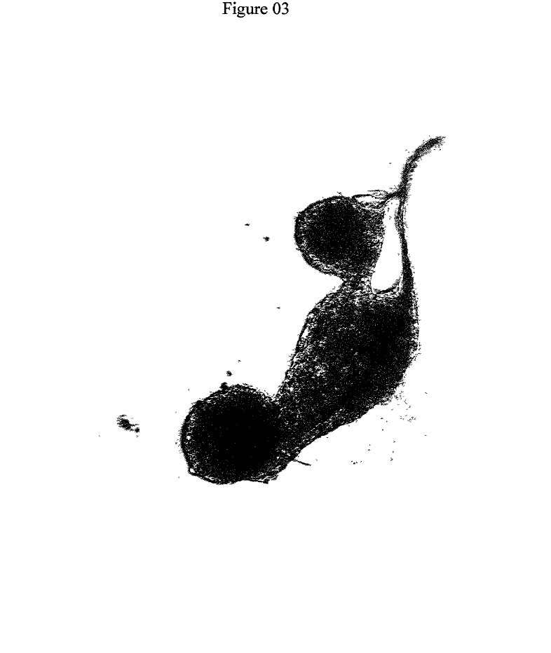

A stealth virus culture was established from a physician who had recently been diagnosed with a prostate cancer. The patient had been practicing as a dermatologist but over the last several years was finding it difficult to continue his practice because of a severe chronic fatigue syndrome-like illness. Multiple pigmented cell clusters developed in his stealth virus culture. A brightly red colored, thread-like structure was seen emerging from one of the clusters. This cluster had also become attached to two smaller pigmented clusters, with the thread-like structure passing close by and seemingly being attached to one of the smaller clusters. It had a thin branch that extended beyond the upper cell cluster. A single cell was seen emerging from the upper left hand side of the largest cell cluster. The appearance of the three cell clusters and the thread-like structure at the beginning of the experiment is shown in FIG. 1. The culture tube was placed between two facing magnets in a Styrofoam holder and examined at various time intervals. By 15 minutes of magnetic field exposure, the single cell coming from the large cell cluster had begun to retract into the cell cluster (FIG. 2). By 12 hours exposure to the magnetic field, the lower cell cluster had become detached from the glass surface and was overriding the lower end of the larger cell cluster. The three clusters have also contracted in size with their diameters being reduced by approximately 20%. They were also noticeably much more pigmented than at the start of the experiment. The small branch from the thread-like structure had also moved towards the upper cell cluster (FIG. 3).

Before placing the tube back into the Styrofoam holder, one of the magnets was removed so that the tube would be exposed to the magnetic field from a single magnet. Major changes ensured over the next two hours. Specifically, the upper small cell cluster broke away from the red thread-like structure and came to overlie onto the upper left hand side of the central cell cluster. The intensity of the pigmentation also increased, as did the amount of fine floating pigmented debris.

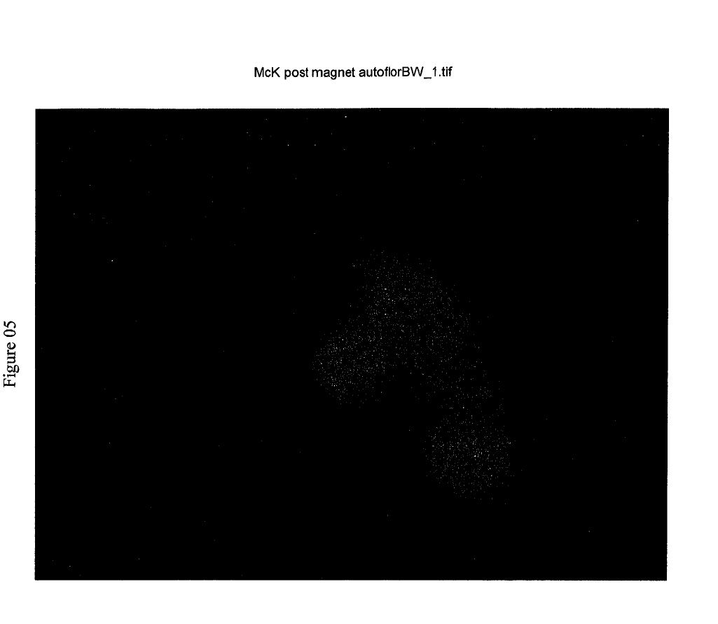

As described in detail in the co-pending patent application concerning light therapy, a striking feature of many of the cell clusters developing in stealth virus cultures is their auto-fluorescence. Although not specifically examined for before beginning exposure to the magnetic field, I had the impression that the clusters would likely give the usual level of auto-fluorescence seen in many of the stealth virus cultures. When I tested for auto-fluorescence at the end of 14 hours of exposure to the magnetic field, it was very striking and easily photographed under dark field illumination (FIG. 5). The clusters also showed a brief red afterglow when the microscope light was turned off. It would seem that exposure to the magnetic field had enhanced the amount and/or the excitation of the auto-fluorescent material within the cell clusters.

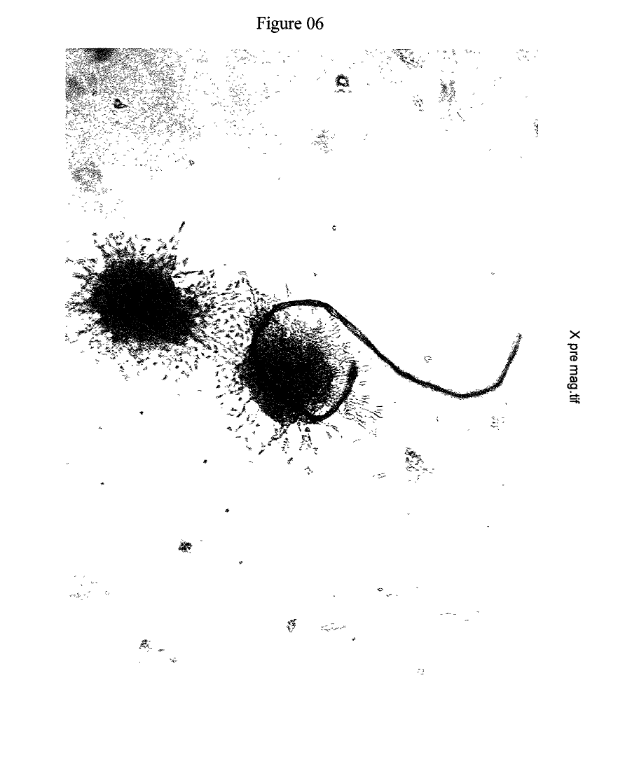

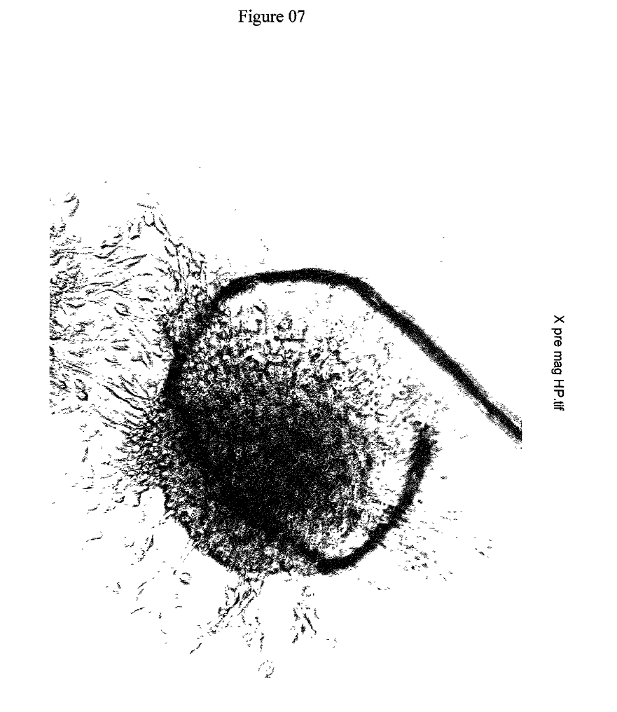

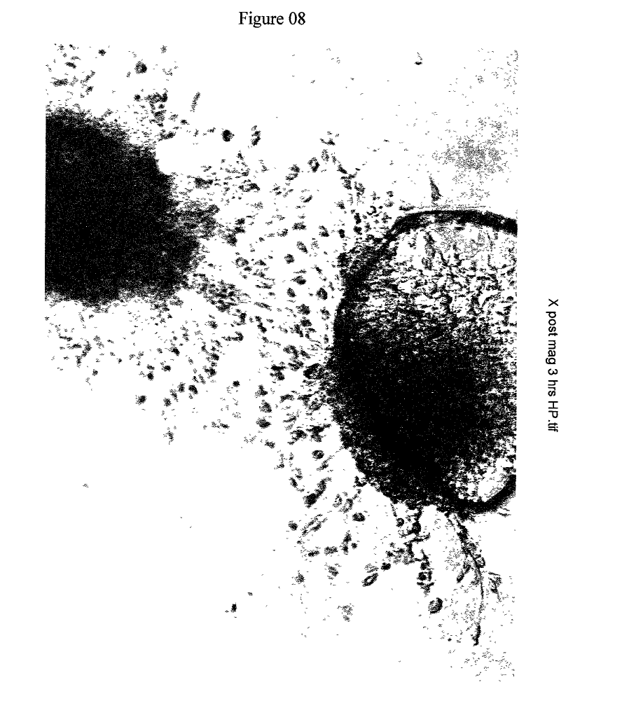

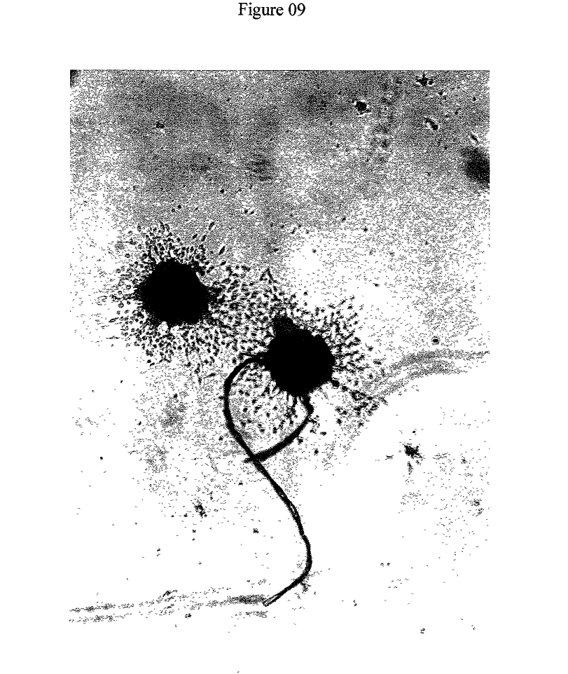

Another stealth virus culture was established. This one came from frozen-thawed bacteria isolated from the stool of a patient with a chronic fatigue syndrome of 10 years duration. Two of the clusters that had formed in the culture are shown at low power in FIG. 6 and at high power in FIG. 7. A long thread-like structure was embedded in one of the cell clusters. Both of the clusters were rimed with relatively normal appearing elongated cells. Following 3 hour exposure to a magnetic field provided by a pair of magnets, many of the cells growing out from cell clusters were showing signs of considerable damage. Specifically, the cells were becoming more rounded and distinct, yet one could no longer easily discern the cell nucleus from the cell cytoplasm (FIG. 8). The magnetic field exposure was continued for another 9 hours using a single magnet. During this time, the diameter of the pigmented areas in each of the cell clusters had decreased by approximately a third. The thread-like structure had flipped over on its side. The cells surrounding the pigmented clusters remained rounded and unhealthy looking and were also becoming slightly pigmented (FIG. 9). Again, as in the earlier culture, there was a marked increase in free floating and glass surface-bound pigmented debris as a result of exposure of the culture to a magnetic field. Several additional thread-like structures also appeared within the culture.

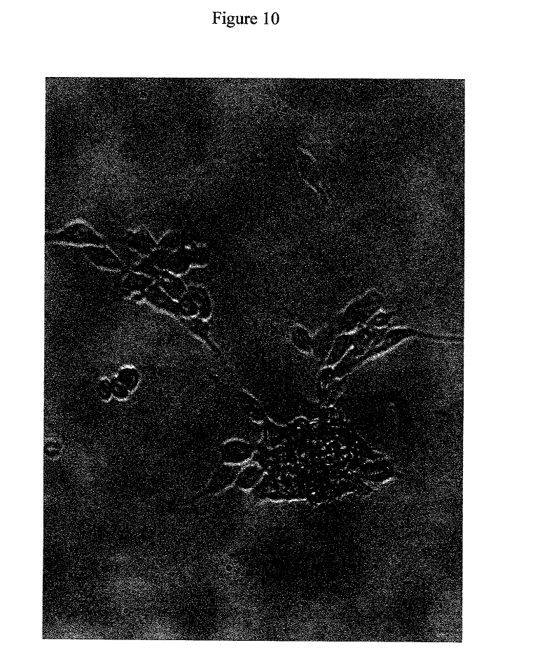

While the most noticeable change throughout the culture was the loss of relatively normal appearing cells around clusters that had acquired more pigmentation, there were suggestions that certain cells had been unaffected by exposure to the magnetic field. By the next day, I could find several cell clusters with very asymmetrical outgrowth of islands of healthy, non-pigmented, and apparently proliferating cells (FIG. 10). Still the major overall effect on the culture from 12 hours exposure to a magnetic field, was the destruction and degeneration of many of the cells.

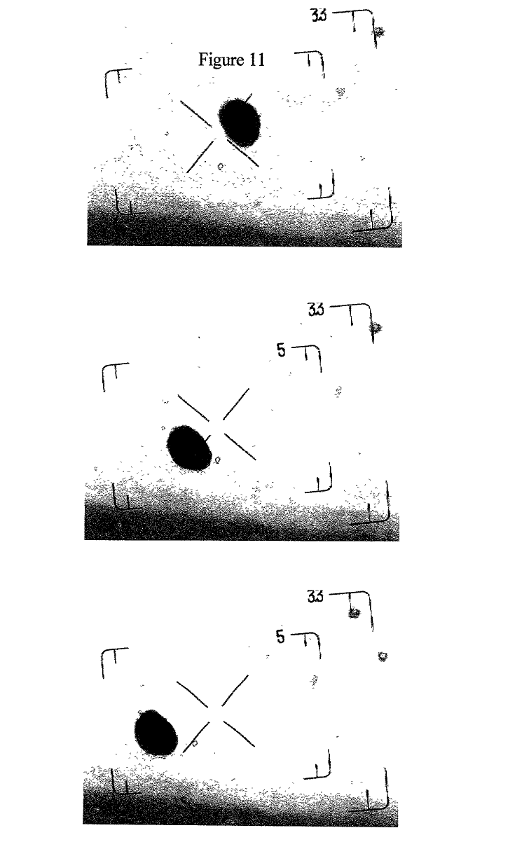

A floating pigmented cluster was microscopically identified in a culture that had been exposed previously to a magnetic field. The cluster was positioned in the center of an incomplete X marking seen in the microscope field of vision. The left hand side of the cluster was noticeably flatter than the right hand side. A hand held magnet was then moved towards the culture tube from the top right hand side of the culture tube. As the magnet approached the tube, the cluster began to move towards the magnet. The upper photograph in FIG. 11 shows the cluster having moved slightly to the right hand side of the X marking. By reversing the magnet so that it now approached the cluster from the lower left side of the culture tube, the cluster rotated 180° and was drawn to the left side of the X marking. (middle photograph in FIG. 11). The flat side of the cluster was now on the right rather than on the left hand side. The cluster continued to be drawn towards the magnet as it was being withdrawn (lower photograph in FIG. 11). The movement of the cluster was readily demonstrated to an independent observer. Discernable magnet induced movement were not seen with most of the clusters examined. This is consistent with the variability of other characteristics of various clusters, such as the extent of pigmentation, formation of thread-like structures, etc. It may also reflect the rather modest strength of the magnet being used (850 gauss) and the approximate 1 cm distance from the magnet to the clusters.

It will be apparent to those familiar with the detection of magnetic and/or paramagnetic materials that far more sensitive methods exist than waving a magnet over the culture tube. The fact that even this crude technique worked on some of the clusters bodes well for the development of sensitive, quantifiable methods to be applied to stealth virus cultures and also to stealth virus infected cells within a patient. Sensitive methods for the detection of magnetic and/or paramagnetic materials in a patient suspected of being stealth virus infected can be used to detect material released from infected cells. Thus, it should be possible to detect stealth virus infected cell-derived magnetic and/or paramagnetic materials in bodily fluids such as urine and saliva as well as stool samples, skin and hair of infected patients. The detection of abnormal magnetic and/or paramagnetic materials in blood units intended for transfusion and in tissue grafts, could provide a useful surrogate marker for the probable presence of a stealth virus infection.

It will also be apparent to researchers interested in the magnetic properties of organic structures that the stealth virus cultures will provide a useful source of materials for detailed molecular studies. Furthermore, it is clear that a magnet can be used to help fractionate the magnetic and/or paramagnetic materials from stealth virus cultures and from stealth virus infected humans and animals. The possible association of the magnetic and/or paramagnetic materials with various metal ions can be approached once purified materials become available.

In a preferred embodiment, one can isolate a stealth virus from blood and also from a tumor sample of a cancer patient. The virus can be grown on MRC-5 human fibroblast cells. The culture is examined for the appearance of a CPE indicative of a stealth virus. The CPE is promoted by daily replacement of the culture medium. The cultures are frequently examined for the accumulation of pigmented materials within cell clusters. The culture is then allowed to stabilize and begin to show signs of repair. This is achieved by not replacing the tissue culture medium. The culture is then exposed to a strong magnetic field and the effects on cell morphology and pigmentation recorded over the ensuring 2-24 hours. If cellular toxicity is induced by the magnetic field, one can confidently arrange for an effective magnetic field to be applied to the patient's in vivo cancer cells. A wide variety of methods exist to expose the cultures to a magnetic field and to administer magnetic therapy to a patient. In particular, one can either use one or more stationary fixed magnets or establish a fluctuating magnetic field using electricity or other oscillating energy sources on a pulsed magnet. The relative efficacy of these different approaches can be established on the cultures from a particular patient and extrapolated to the design of the in vivo therapy. The intensity of the in vivo magnetic exposure can also be based on the in vitro culture findings and also on the desired depth of effective magnetic field penetration. The in vivo therapy can be targeted directly to a known site of a stealth virus infection, such as a stealth virus infected cancer, or can be administered more systemically to sites expected or suspected of harboring infected cells.

The effect of magnetic field therapy on tumor size, consistency and other changes can be determined and used to schedule the length, intensity and frequencies of subsequent magnetic therapy sessions. Progress can also be ascertained by conducting fine needle aspirates (FNA) of the tumor before and after magnetic therapy sessions and ascertaining evidence for tumor cell death. FNA derived material can also be cultured for residual evidence of stealth virus infection. Beneficial effects on a local tumor will encourage the use of similar magnetic therapy to empirically destroy potential metastatic cancer deposits throughout the body. Thus magnetic therapy can be extended to body sites suspected of possibly containing cancer but in which no actual tumor mass has been identified. These sites can include the circulating blood. The effect of magnetic field exposure on subsequent stealth virus cultures obtained from a treated patient can also be used as a marker for the efficacy of therapy.

Stealth virus can reside in bowel bacteria. Magnetic field mediated removal of infected bacteria from the body may also prove useful at reducing virus burden and potentially toxic bacterial products. Blood products and tissue grafts could also be treated with magnetic field therapy as a means of destroying any stealth virus infected cells in which there is magnetic and/or paramagnetic material.

In administrating systemic magnetic field therapy to certain stealth virus infected patients, an effort may need to be made to shield the brain from the therapeutic magnetic field source. This issue is under consideration since intense intracranial magnetic field exposure might have a potentiating effect on any ongoing stealth virus induced damage to brain cells. For this reason, I am currently not suggesting the use of magnetic field therapy for stealth virus infected patients with advanced degenerative neurological illnesses.

Another theoretical adverse effect of magnetic field-mediated systemic stealth virus therapy, is the possible induced alterations in a coronary artery lesion that involves the active presence of a stealth virus infection. At the present time, intense magnetic exposure to the heart in a stealth virus infected patient suspected of having coronary artery disease, may, therefore, not be advisable. Alternatively, it may turn out that magnetic exposure to a stealth virus associated coronary artery lesion may destroy sufficient stealth virus infected cells to allow healing to occur. Resolutions of this and other issues will come from clinical trials on the use of magnetic field therapy on stealth virus infected patients.

While the therapy of stealth virus associated cancer is the most obvious application of the detection of magnetic and paramagnetic material in a stealth virus infected cell, the methods described can be applied to treating humans and animals manifesting other types of clinical illness resulting from a stealth virus infection. It is known that stealth viruses can produce widespread multi-system damage affecting various organs and body systems. Stealth virus infected patients may have endocrine, immunological, metabolic, auto-immune and other disorders (1,2). Although the pathological basis for how stealth viruses are inducing these diseases is not fully understood, it would be reasonable that at least under some circumstances additional damage to infected cells might actually improve the clinical condition by removing possible sources of toxic and/or antigenic materials. The judicious use of magnetic field therapy in such patients could help resolve this issue. Certainly, stealth infected cell destructive magnetic field therapy could delay the further progression of disease due to continued viral replication in infected cells.

I can, therefore, conclude that a strong magnetic field is toxic for cells from certain stealth virus cultures. The toxicity can be ascribed to the presence of magnetic and/or paramagnetic intracellular materials in many of the stealth virus cultures examined. The discovery of this material, along with the finding of magnetic field induced cellular toxicity of stealth virus infected cells, has provided a clear rationale for magnetic based therapy for stealth virus infected humans and animals. The initial clinical experience of using a magnet in a stealth virus positive patient with breast cancer is encouraging.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Additional advantages and modifications will readily occur to those skilled in the art. Variations and changes may be made without departing from the spirit of the invention

What I claim as my invention is:

1. A method to test for the ability of a stealth virus isolated from a patient to induce the production of magnetic and/or paramagnetic material in cells, comprising i) culturing blood and/or tissue samples from a stealth virus infected patient in a manner that will allow for stealth virus induced production of pigmented intracellular and/or extra-cellular material, ii) determining microscopically whether freely floating pigmented material will move either towards or away from an approaching magnet, and iii) concluding that magnetic and/or paramagnetic material is inducible by the stealth virus if the pigmented material moves in response to the approaching magnet.

2. The method of claim 1 in which the detection of magnetic and/or paramagnetic material in a stealth virus culture is achieved by using an alternative detection method.

3. A method of testing the ability of a magnetic field to cause damage to stealth virus infected cells comprising i) culturing blood and/or tissue samples from a stealth virus infected patient in a manner that will allow for the development of a stealth virus induced cytopathic effect ii) exposing the culture to a magnetic field and iii) examining the cultured cells microscopically for morphological and/or functional signs of cell damage in response to being exposed to the magnetic field.

4. A method of treating a stealth virus infected patient based on exposing known or suspected virus infected cells within the patient to a magnetic field that can be shown to induce cellular damage to stealth virus infected cells in tissue culture, and not to cause similar morphological and/or functional signs of cell damage to corresponding normal uninfected cells.

5. The method of claim 4 in which the stealth virus infected cells are causing diseases in animals.

6. The method of claim 4 in which stealth virus infected cells in the patient are present within a cancer.

7. The method of claim 6 in which the therapy is applied to a known cancer mass present within the individual to be treated.

8. The method of claim 6 in which the therapy is applied to an area of the body in which one might expect or suspect microscopic deposits of cancer cells to be present.

9. The method of claim 4 in which the stealth virus infected cells are causing a non-malignant illness in a patient.

10. A method of treating a stealth virus infected patient based on exposing stealth virus infected bacteria within the patient to a magnetic field that is capable of killing cultured bacteria infected with the stealth virus derived from the patient.

11. The method of claim 10 in which the stealth virus infected bacteria are causing a bacterial illness.

12. A method of destroying all or some of any suspected stealth virus infected cells in a blood sample, or in a blood unit to be used for transfusion, or in other biological samples, comprising the use of a magnetic field administered to the sample in such a manner that has previously been shown to cause the destruction of at least a proportion of the stealth virus infected cells in other blood samples and/or blood units that were known to be infected with a stealth virus.

13. A method of claim 12 in which the stealth virus infected cells are suspected of being present in a tissue graft to be used for transplantation.

14. A method of destroying all or some of any suspected stealth virus infected bacteria in an environmental sample, comprising the use of a magnetic field administered to the sample in such a way that it can be shown to cause the destruction of at least a proportion of the stealth virus infected bacteria in a comparable environmental sample that was known to be infected with a stealth virus.
